(19) United States
(12) Patent Application Publication
(10) Pub. No.: US 2006/0058590 A1
(43) Pub. Date: Mar. 16, 2006

(54) Method and system for assaying agitation

(76) Inventors: Geoffrey M. Shaw, Christchurch (NZ); James Geoffrey Chase, Christchurch (NZ)

Correspondence Address: Stephen M. De Klerk;BLAKELY, SOKOLOFF, TAYLOR & ZAFMAN LLP, Seventh Floor, 12400 Wilshire Boulevard, Los Angeles, CA, 90025, US

(21) Appl. No.: 10/925,395

(22) Filed: Aug. 24, 2004

(51) Int. Cl. A61B 5/00

(52) U.S. Cl. 600/301

(57) ABSTRACT

A method of physiologically quantifying patient agitation presented is based on reliable, objective physiological signals. The present invention is capable of quantifying autonomic nervous system interactions to provide an objective measurement of agitation. Adaptive autoregressive (AR) signal processing techniques are used to analyze heart rate (HRV) and blood pressure (BPV) variability and are combined with a fuzzy quantifier to measure agitation levels. Results show that agitation in normal subjects can be assessed and quantified using this approach, including differentiating periods of calm. Additionally, it has been shown that detected periods of agitation in ICU patients correlate well with subjective assessment by trained medical staff using the modified Riker SAS and with the objective assaying of patient motion. These results show that agitation can be quantitatively measured and assessed using common biomedical signals. Finally, agitation induced in normal subjects correlates well to agitation in ICU patients, as both show similar changes in the measured biomedical signals during agitated periods.

### TECHNICAL FIELD

The present invention relates to a method and system for assaying agitation, particularly in clinical applications.

### BACKGROUND ART

Patient agitation prolongs recovery, interferes with administration of drugs and therapeutic procedures, and decreases the safety of the patient and medical staff. While sedation is administered to maintain patient comfort, in the Intensive Care Unit (ICU) most sedation is administered in addition to this amount in response to patient agitation [Fraser et al 2001]. The estimated yearly cost of ICU administered sedatives and/or analgesics in the US is US $0.8-1.2 billion [Kress et al, 2000]. However, current methods of assessing agitation are subjective and prone to error leading to over-sedation, and increases in cost and length of stay [Kress et al 2000; Jacobi 2002; Wiener-Kronish 2001]. Therefore, a consistent, quantifiable, physiologically-based method of measuring agitation that enables more effective sedation administration could save significant drug and resource cost, reduce patient stay, and improve health care.

Agitation can result in dangerous situations for both the patient and intensive care staff. Among the most common risks are over-sedation and accidental exturbation, i.e. removal of the endotracheal tube, which can immediately endanger the patient's life. There are also risks for intensive care staff who must restrain the most combative patients, making their work more difficult, and limiting time for the care of other patients.

Over-sedation is also a risk given the long-term continuous infusions given to critical care patients to control agitation. However, continuous intra-venous (IV) infusions lead to prolonged sedation for a number of reasons.
Patients rapidly become tolerant to some of the most common frequently administered sedatives (e.g. benzodiazepines), thus requiring more sedative to achieve the same effect.
The half time decrement of these sedatives is reduced when administration is prolonged, resulting in an extended duration of effect. Therefore, the frequent use of continuous infusions of these medications in the ICU, primarily in response to agitation, has been found to lead to over-sedation and the need to administer ever-increasing quantities of these medications [Jacobi 2002; Wiener-Kronish 2001]. In contrast, it was also found that a simple protocol of shutting off of sedation infusions every morning until agitation manifests reduced the sedation administered and cut length of patient stay by 33% [Kress et al 2000].

There are numerous subjective sedation-agitation assessment scales. Some of the most common include the: Ramsay Scale [Fraser et a/2001; Jacobi 2002; Szokol et a/2001], Riker Sedation-Agitation Scale (SAS) [Fraser et al 2001; Riker et al 1999], Motor Activity Assessment Scale (MMS) [Kress et al 2000; Cohen 2002], Richmond Agitation-Sedation Scale (RASS) [Sessler et al 2002], Vancouver Interaction and Calmness Scale (VICS) [de Lemos et al 2000] and Glasgow Coma Scale [Szokol et al 2001; Carrasco 2000]. All of these scales depend on subjective, qualitative assessment of patient movement or the patient's auditory and visual ability. A further limitation is that they often provide multiple criteria for each agitation level. Hence, the patient may exhibit behavior that meets the criteria of more than one level, making it difficult to correctly identify the degree of agitation. Furthermore, many sedation-agitation scales do not allow for situations where the patient may be sleeping or sedated but react violently to stimulation. Such patients would be classified in one of the sedation classes and it is left to the nursing staff to remember the excessive response, often leading to inconsistencies in agitation control and sedation management [Sessler et al 2002]. Moreover, the reliance of these scales on subjective assessment criteria, rather than quantifiable, measurable data, creates several avenues for undesirable inconsistency and variability in the agitation grading and hence, sedation administration. A consistent measure would enable more consistent and significantly improved agitation and sedation management via automated or semi-automated methods, as has been shown in simulation [Shaw et al 2003].

Research concerning these rating scales has also shown that a considerable number of nurses believe that due to the large intra-patient and inter-patient variability of patient sedation requirements, only an experienced nurse, who often reassesses the patient's needs with their own methods, is able to deliver appropriate care [Weinert et a/2001]. The result is inconsistent inter-nurse assessment and treatment of patient agitation. Furthermore, even if all nurses used the same method and guidelines for assessing agitation, their individual judgment may still be influenced by their personal expectations and patient history. Patients who lie quietly without moving, have neuro-muscular blockade, or are unable to communicate would exacerbate this problem, preventing any significant agitation assessment with said scales. Such difficulties are not confined to the ICU, but are also a significant problem in pediatric critical care units.

The manifestation of agitation is not confined to hospitals or other medical environments. Individuals may exhibit agitation or other personal displacement gestures in stressful situations such as during police or customs questioning, employment interviews, driving, flying and so forth.

In such non-medical environs any form of agitation assaying is typically either wholly absent or if present, consists of a subjective, qualitative system such as a policeman's visual observation and written notes. Such procedures are clearly prone to inaccuracies and variations between individuals.

There is thus a need for a quantitative, objective assaying of an individual's level of agitation. Particularly in medical environs

It is an object of the present invention to address the foregoing problems.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

According to one aspect of the present invention there is provided an objective method of assaying agitation in an individual or patient, said method including;
automated monitoring of at least one metric of
   a patient's autonomic nervous system (ANS);
   expert systems or rules delineating other clinical events from agitation (eg atrial fibrillation from large spikes in HR due to agitation) and/or
   physical movement of one or more defined region(s) of interest (ROI) of the patient's body,
performing signal processing on physiological signals associated with the monitored metric and
calculating agitation from changes in said processed physiological signals.

Preferably, said agitation calculation provides a corresponding agitation value within a defined agitation index.

According to a further aspect of the present invention there is provided a system for objective assaying of agitation in an individual subject or patient, said system including;
automated monitoring apparatus capable of monitoring at least one metric of
   a patient's autonomic nervous system (ANS);
   expert systems or rules delineating other clinical events from agitation (eg atrial fibrillation from large spikes in HR due to agitation) and/or
   physical movement of one or more defined region(s) of interest (ROI) of the patient's body,
   and/or
   physical movement of one or more defined region(s) of interest (ROI) of the patient's body,
signal processing means capable of processing physiological signals associated with the monitored metric and
processing means capable of calculating agitation from changes in said processing physiological signals.

Preferably, said agitation calculation provides a corresponding agitation value within a defined agitation index.

The present invention is described herein with reference to agitation in a medical patient (in particular critical care patients in ICU), though it will be appreciated that the invention is not necessarily restricted to same. Thus, the term ‘patient’ is used herein in its broadest sense to include any individual or subject being monitored for agitation and is not restricted to medical or clinical applications or environments.

Preferably, said physiological signals include;
heart rate variability (HRV);
blood pressure (BP);
blood pressure variability (BPV);
respiratory rate (RR);
heart rate derivative (HRD);
blood pressure derivative (BPD);
temperature;
cardiovascular metrics, including cardiac output (CO), diastolic blood pressure, cardiac filling volumes;
EEG/brain wave measurements;
physical movement of one or more defined regions of interest (ROI) of the individual's body.

The ANS includes both the sympathetic nervous system (SNS) and parasympathetic nervous system (PNS). According to one aspect of the present invention, patient agitation can be measured by determining the amount of SNS activity present in readily measurable available physiological signals such as HRV, BP and/or BPV; as a patient manifests agitation, the SNS response to this stress and any resultant ROI motion generates changes in these physiological signals. Since these signals are commonly used for analyzing patient sympathetic and parasympathetic nervous system interactions and are readily available from ICU patients, they can therefore provide good indicators of patient agitation ICU patients (Bianchi et al 1997, Lombardi et al 1987, Mainardi et al 1997). More specifically, as agitation manifests heart rate and blood pressure have been observed to rise. These increases lead to decreased HRV, and elevated BP and BPV levels (Pfister et al 2001).

It will be noted that although HRV and BPV signal are functions of the ANS response to stress, the manifestation of excessive motion, even if based on a central nervous system (CNS) function, will also result in changes in ANS function. In a sedated ICU patient CNS (cognitive) function is unknown, and therefore ANS changes with their impact on the cardio vascular system (CVS) may be used as appropriate surrogates that accompany the excessive motion found in patient agitation.

Thus, by measuring said physiological signals and determining to what level and in what manner they correlate with the objectively assessed agitation, a consistent, quantifiable measure of patient agitation can be created for each signal. A quantified measure of patient agitation also offers a platform for further understanding and quantifying the effects of different sedative therapeutics in reducing patient agitation.

Although the monitored cardiovascular signals may be used in conjunction with analysis of the patient's ROI movement, each technique is initially discussed separately herein.

Thus, according to a further aspect, the present invention includes an objective method of assaying agitation in an individual, said method including;
an automated monitoring of at least one metric associated with physical movement of one or more defined region(s) of interest (ROI) of the individual's body, and comprises the steps:
   image capture of at least one ROI;
   determination of motion in a ROI;
   quantification of relative patient agitation.

Preferably, said determination of motion in ROI step further includes
determination of power spectral density (PSD).

Preferably, said method includes the further step of
calculating a corresponding agitation value within a defined agitation index using a fuzzy logic inference system.

Considering the stages in more detail, the individual patient's body is subdivided into defined regions of interest (ROI) according to the primary body portions likely to exhibit movement, e.g. in the case of a supine bedded patient, the patient ROI are the patient's limbs and head.

It will be appreciated however that the present invention also includes the configurations where a captured image frame may contain only a single ROI and which may be coterminously dimensioned with the border of the captured image.

Preferably, said determination of motion distinguishes between patient body motions and third party individuals. Said third parties may include nursing of medical staff, patient relatives or the like.

Preferably, said at least one third party ROI are provided about the periphery of the captured image.

In one embodiment, movement detected in a third party ROI and subsequently detected in an adjacent patient ROI, causes the motion reading from the patient ROI to be de-weighted until the movement ceases.

According to one embodiment, the automated monitoring apparatus includes an image detector, e.g. a digital video or stills camera.

Preferably, the system determines a normalized measure of motion power for both the patient ROI regions and third party ROI regions.

Preferably, said motion determination is performed using block comparison algorithm. A block comparison algorithm captures and quantifies movement by calculating the differences between pixels or blocks of pixels in successive frames to ensure minimal computational intensity.

Preferably, said block comparison algorithm provides a single scalar index P(t), given by:

$$P ( t ) = {\sum }_{x - 1}^{m} {\sum }_{y = 1}^{n} {Dt ( x , y )}^{2}$$

calculated from the sum power difference over successive captured image frames.

Preferably, P(t) is normalized with respect to the maximum attainable P(t) value.

In an alternative embodiment, said motion determination is performed utilizing normalized correlation coefficients to measure change between captured image frames, or between ROI images.

Preferably, said correlation coefficient rₖ between captured image frames for a given region k is given by

$${r}_{k} ( t + 1 ) = \frac{{\sum }_{xy} \{ ( {f}_{t} ( x , y ) = {\overline{f}}_{t} ) ( {f}_{t} + 1 ( x , y ) - {\overline{f}}_{t} + 1 ) \}}{\sqrt{\{ {\sum }_{xy} {( {f}_{t} ( x , y ) - {\overline{f}}_{t} )}^{2} \} \times \{ {\sum }_{xy} {( {f}_{t} + 1 ( x , y ) - {\overline{f}}_{t} + 1 )}^{2} \}}}$$

where fₜ(x,y) is a pixel intensity value at location (xy) at time, t, and \overline{f}ₜ is the average pixel value over the entire region, k, with corresponding definitions for time t+1. The numerator is the covariance between frames for that region and the denominator is the combined variance.

The correlation coefficient rₖ equation presents a direct, normalized measure of the change between image frames, presenting a clear measure of the level of motion. Therefore, bias due to changes in lighting or differences in camera distance or position that can influence the block comparison method is eliminated. The magnitude of rₖ(t+1) approaches 0 when there is excessive motion because the covariance between frames is very low, and conversely approaches 1.0 when there is little motion. It will be appreciated that this value can be determined (according to the definition of k) for the entire patient regions and nurse areas or combined over selected ROI.

Mathematically, the value of rₖ can vary between −1 and +1, depending on the change in motion. However, the magnitude of the motion may be measured by the variance between frames, and thus represented by the coefficient of determination.

Preferably, the coefficient of determination, Rₖ=r²ₖ, over the range from 0 to +1, eliminating the phase shift information in the sign. As a result, a motion-related agitation index can be defined as Aₖ (t+1)=1−rₖ(t+1)²=1−Rₖ(t+1), where k is defined for the nursing edge region ROI (8-11), and/or specified patient ROI. Therefore, Aₖ approaches 0 when Rₖ approaches 1 and the motion is very low between frames. Similarly, Aₖ approaches 1 during extensive motion.

Fuzzy mathematics is an apt tool for classification and diagnostics problems where the dynamics of the system are not well known. Fuzzy system models rely on rules defined from logic built from observational data to approximate the unknown dynamic behavior. In one embodiment, the dynamics are defined to range between 0 and 1 as a convenient decimal percentage. The result is a fixed neural network model that is derived from the fuzzy mathematics and rules defined, providing a measure of probabilistic likelihood of each membership function of a fuzzy set representing the likelihood of different levels of agitation (e.g. low, medium, high).

Preferably, the present invention utilizes fuzzy mathematics to calculate a single motion-related agitation index from the captured image frame-to-frame correlation coefficients rₖ for both patient and third-party ROI motions.

Preferably, for a patient under medical supervision by a nurse, a patient agitation value on said agitation index is given by at least one of the following rules, wherein;

| Rule | Patient-motion | Nurse-motion | agitation |
|---|---|---|---|
| 1. | low | low | low |
| 2. | medium | low | medium |
| 3. | high | low | high |
| 4. | low | medium | low |
| 5. | medium | medium | high |
| 6. | high | medium | high |
| 7. | low | high | medium |
| 8. | medium | high | high |
| 9. | high | high | high |

It will be appreciated that noise may be reduced in the captured images by higher frame rates, additional filtering for root mean square (RMS) or moving average values, and/or longer multi-frame time period windows, rather than immediate frame-to-frame calculations.

As discussed above, agitation may also be assayed from monitoring physiological metrics including cardiovascular and respiratory as well as patient motion.

Thus, according to one embodiment, said automated monitoring of at least one metric of a patient's autonomic nervous system (ANS) includes monitoring power spectral density (PSD) of both HRV and BPV. The HRV tachogram examines the R-R interval between heart beats, and the BPV systogram examines the changes in systolic blood pressure.

Preferably, said steps of quantifying agitation include;
(QRS peak detection and R-R interval calculation) and/or (systolic and diastolic blood pressure values detection)
spectral estimation and calculation of PSD in VLF, LF, and HF frequency bands and
determination of patient agitation from changes in signal dynamics

Preferably, said ORS peak detection and R-R interval calculation performed in an ECG signal can be easily detected using a Haar wavelet.

Preferably, said spectral estimation and calculation of power in VLF, LF, and HF frequency bands is preformed using frequency domain analysis, preferably in the frequency bands high (HF) 0.15-0.4 Hz; low (LF) 0.07-0.14 Hz; very low (VLF) 0.0033-0.04 Hz.

In one aspect, said spectral analysis of R-R and/or systolic blood pressure signals is performed using an adaptive autoregressive (AR) spectral estimation method.

Thus, in a preferred embodiment, said PSD, P_{AR}, is given by:

$${P}_{AR} = T {σ}_{∞} {\vert \frac{1}{A ( f )} \vert}^{2}$$

where T is the sampling interval used for scaling and |A(f)| is the frequency response obtained from the AR coefficients aᵢ. Using the fast recursive least squares (RLS) algorithm enables an update of the spectral estimation every time a new sample is available (Marple 1987).

Preferably, said determination of patient agitation from changes in signal dynamics is determined using a fuzzy-logic inference system (FIS).

After estimating the PSD, the spectral power in the VLF, LF and HF frequency bands are calculated. Preferably, inputs of said FIS include the HRV ratio VLF/HF and the BPV ratio HF/VLF. These signals measure the decrease in HRV and increase in BPV, respectively, as agitation manifests. Hence, both ratios are expected to rise when agitation occurs.

Four FIS measurements were used for each ratio input signal; the current signal value (V1) and its mean value over the prior 5, 10 and 20 min (V5, V10, V20). These values were chosen based on clinical expertise and the action time of the sedatives used (3-10 min). Essentially, these time periods represent instantaneous (1), immediate (5), sedative effect time (5 and 10) and long term (20) states of patient agitation. It will however be appreciated alternative time period increments may be chosen. This technique allows changes in the signal to be followed and facilitates the detection of longer-term trends.

Preferably, individual agitation levels for each input signal are recorded at a plurality of time increments T2, T3, T4, . . . Tn preceding an instantaneous level T1, wherein the individual agitation levels, obtained for HRV, systolic blood pressure and BPV, are then combined in create a single agitation value according to the rules:

| Rule | T1 | T2 | T3 | T4 | Agitation |
|---|---|---|---|---|---|
| 1 | Low | — | — | — | Low |
| 2 | Medium | High | — | — | Low |
| 3 | Medium | Medium | Low | Low | Low |
| 4 | Medium | Medium | Medium | Medium | Low |
| 5 | Low | Low | Low | Low | Low |
| 6 | High | High | High | High | High |
| 7 | High | Low | Low | Low | High |
| 8 | High | Medium | Low | Low | High |
| 9 | High | Medium | Medium | Medium | Medium |

According to a further aspect, the present invention provides a method of sedation administration including the steps;
objectively quantifying agitation according to the method substantially as described above;
inputting said quantified agitation to an automated sedation administration system,
administering defined quantities of one or more sedatives in proportion to said quantified agitation.

The present invention also provides a system for sedation administration including;
said system for objectively quantifying agitation substantially as described above;
an automated sedation administration system capable of receiving said quantified agitation and administering defined quantities of one or more sedatives in proportion to said quantified agitation.

By providing a quantified measure of the agitation of a patient, accurate sedation administration becomes a viable clinical capability with significant consequential improvements on patient care and cost reduction. Sedation infusion pumps and other sedation administration systems are known but currently are used to sedate the patient according to settings derived from nursing/medical observations of the patient's physiological metrics and visible displays of agitation.

In a yet further embodiment, the above method and system for quantifying agitation may be incorporated in an alarm system particularly for use in non-ICU environments to alert nursing staff should a patient's agitation exceed a predetermined threshold value.

Thus, the present invention provides a method of alerting nursing/medical personnel to excessive patient agitation, including the steps;
monitoring agitation in accordance with the above-described methods;
outputting an alarm signal when said quantified agitation exceeds one or more predetermined threshold values.

According to a further embodiment, the present invention may be used to provide user fatigue and/or agitation monitoring method and system characterised in that when a user's physical movement from one or more ROI exceeds one or more upper or lower movement threshold levels, a signal is output to one or more systems including:
an audible and/or visual alarm signal,
a graphical and/or alphanumeric information display,
one or more direction and/or velocity control means of a vehicle,
audio system,
data-logging means.

Thus, in an example of vehicle driver fatigue, a drowsy driver may provide numerous changes in motion detectable in one or more specifically defined ROI such as:
reduced eye movement, indicating. reduced blinking and/or scanning;
increased mouth movement indicating possible yawning;
reduced arm and leg/foot movement indicating reduced steering and/or speed control;
increased head movement indicating possible lolling of the user's head during loss of consciousness episodes.

Such signals may be used by the system to provide a visual alert to the driver, such as a flashing light and/or alarm signal, to increase the volume of an audio system (e.g. increasing the radio volume) or include active safety measures such as reducing the vehicle's speed and/or sensitivity to steering input to mitigate the effects of a potential crash. It will be appreciated that numerous alternative actions are possible without departing from the scope of the invention.

In further refinements, the incorporation of vehicle location means such as GPS units and digital cartography enable the system to reduce the alarm threshold sensitivities according to the type of road being traveled, e.g. less movement is expected on motorways and major roads in comparison to minor, twisty roads.

According to a further aspect, the present invention provides a means of quantifying user agitation during non-medical assessment environments such as during police questioning, and the like, wherein agitation quantified using the above-described methods is compared to established data recorded for non-stressed individuals to provide a relative agitation index.

Whilst not in itself an unequivocal indication that the subject may be stressed or lying during questioning, it nevertheless provides a further quantitative information source for authorities to evaluate the voracity of the subject's statements.

### BRIEF DESCRIPTION OF DRAWINGS

Further aspects of the present invention will become apparent from the following description which is given by way of example only and with reference to the accompanying drawings in which:

FIG. 1 shows an captured image frame subdivided into regions of interest (ROI) according to a first preferred embodiment of the present invention;

FIG. 2 shows patient motion fuzzy set membership functions where the x-axis is the patient input motion coefficient and the y-axis is the agitation level, according to the first preferred embodiment;

FIG. 3 shows a fuzzy transfer surface relating nurse and patient motion to patient agitation according to the embodiment shown in FIGS. 1 and 2;

FIG. 4 shows motion power level for different types of agitated motion according to a further preferred embodiment;

FIG. 5 shows a first patient and nurse correlation coefficients and resulting agitation index for a selected 30 minute period, according to a further preferred embodiment;

FIG. 6 shows a second patient and nurse correlation coefficients and resulting agitation index for a selected 30 minute period, according to a further preferred embodiment;

FIG. 7 shows a third patient and nurse correlation coefficients and resulting agitation index for a selected 30 minute period, according to a further preferred embodiment;

FIG. 8 shows results from the patient results shown in FIG. 7 with a variety of agitation metrics and combined agitation score at a different time frame to that shown in FIG. 7;

FIG. 9 shows an HRV (a) and systolic BP (b) for a normal subject during CWT and CP tests, according to a further preferred embodiment;

FIG. 10 shows an HRV (a) and systolic BP (b) for a further normal subject during CWT and CP tests, according to a further preferred embodiment;

FIG. 11 shows a FIS output for HRV (a) and BPV (b) for an ICU patient;

FIG. 12 shows a FIS output for HRV, BPV and systolic BP values (a) for the ICU patient in FIG. 11 and resulting overall agitation level (b), and

FIG. 13 shows a FIS output for HRV and BPV values (a) for a second ICU patient and resulting overall agitation level (b).

### BEST MODES FOR CARRYING OUT THE INVENTION

The present invention provides an objective method and system of assaying agitation in an individual, particularly critical care patients such as those in ICU. The quantification of agitation may be derived from automated monitoring of either at least one metric of an individual's autonomic nervous system (ANS) and/or physical movement of one or more defined regions of interest (ROI) of the individual's body. Although direct benefits may be gained from the use of both monitoring methods, both are considered individually herein in more detail.

Patient movement currently plays at least the primary, if not entire, role in the assessment of patient agitation when the patient is reasonably sedated [Weinert at al 2001]. This dominant role is reflected in a study carried out to investigate nurses' assessment of movement and agitation in sedated patients [Foster et al 2001]. Hence, current agitation assessment can be dominated by the assessment of excess or undesirable patient motion. Therefore, by measuring the power in patient motion over different time intervals, the present invention provides a relative yet objective patient agitation index.

This approach can be extended to include multiple motion signals representing motion of different portions of the body, the limbs (arms, legs) and head in particular for this case. In typical nursing conditions for sedated and/or critical care patients such as ICU, the patient will receive routine monitoring and nursing care, together with nursing intervention in the event of agitation manifestation. Thus, to be effective the system must also be capable of differentiating between motion of the patient and that of the nursing staff motion working with that patient.

A fuzzy inference system (FIS) is used in a preferred embodiment to differentiate between the patient motion and nursing/medical staff motion. The level of agitation is then classified by using medical experience and extensive observation to create rules from which a patient agitation level can be quantified. A FIS is particularly apt for this role as the system dynamics of sedated patient agitation are essentially unknown. Clinically, a quantified measure of patient agitation also offers a method of improving sedation administration, as well as a platform for quantifying the effects of different sedative therapeutics in reducing patient agitation.

In a preferred embodiment, the present invention quantifies agitation by monitoring the physical movement of at least one ROI including the steps of
ROI image capturing,
motion determination in the ROI,
optionally determining the power spectral density (PSD),
qualifying the relative patient agitation and preferably calculating a corresponding agitation valve within a defined agitation index using a FIS.

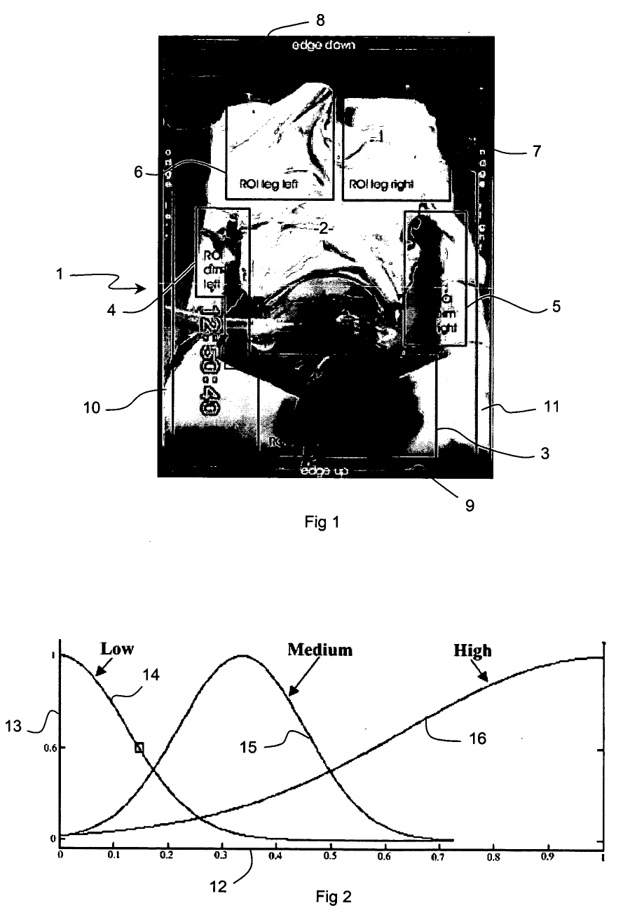

FIG. 1 shows a captured image (1) of a simulated ICU patient (2) with a plurality of ROI defined to cover the head (3), left arm (4), right arm (5), left leg (6, and right leg (7). In addition to patient-specific ROI, further edge regions ROI labeled “edge down” (8), “edge up” (9), “edge left” (10) and “edge right” (11) are monitored at the upper, lower, left and right peripheral screen edges respectively, to detect external third party movement. This is primarily due to medical staff interacting with the patients but may possibly include patient relatives or the like. When movement in the nursing edge ROI (8-11) outside the patient ROI is detected, and the movement subsequently moves into an adjoining patient ROI (3-7), the resulting reading in the affected ROI is appropriately de-weighted until such time as the movement ceases. More specifically, the system segments the image into patient and nursing (edge) regions (8-11) and determines a normalized measure of motion power in each. The monitoring method can also be adapted to re-define the body area of an ROI and/or select/de-select specific ROI to individually examine motion of specific body parts or areas of the patient.

Image capture may be preferred by any suitable electro-optical device such as a video or stills, digital camera, thermal imager or the like. The captured image (1) shown in FIG. 1 is a single still from a digital camera (not shown) taken at a continuous rate of five frames per second (fps). The captured images (1) are recorded in any convenient format (e.g. AVI, MPG, WMV, ASF, RAM, etc) and stored on a PC (not shown) or similar digital storage means. In one embodiment, the captured images (1) are then converted and stored as bitmap images of 320×240 pixels (4/3 format) on the PC to facilitate image processing. As the main image criteria is detection of relative movement between individual images, the image quality may be reduced from its potential maximum to minimize computational intensity and processing, e.g. by converting the bitmap from a color (24-bit) image to an 8-bit grey-scale image.

In one embodiment, motion detection is performed using block comparison methods [Shaw et al 2003; Lam et al 2003]. A block comparison algorithm captures and quantifies movement by calculating the differences between pixels or blocks of pixels in successive frames to ensure minimal computational intensity. Since subtraction is computationally simple, this technique provides efficient data processing, enabling real-time implementation. The intensity values resulting from the subtraction can then be further filtered or processed according to the specific conditions of the application. By comparing the results over multiple frames, it is possible to detect and quantify the magnitude of specific body part movements over time.

If fₜ is a frame that occurs in time t, with 8-bit (0-255) greyscale pixel values, fₜ(x,y), located at (x,y), the pixel difference Dₜ at times t+1 and t is defined as:
Dₜ(x,y)=fₜ+1(x, y)−fₜ(x, y) (1)

The sum power difference over the frame is therefore defined as:

$$P ( t ) = {\sum }_{x = 1}^{m} {\sum }_{y = 1}^{n} {{D}_{t} ( x , y )}^{2} \qquad ( 2 )$$

where P(t) is a single scalar index that can be used to compare frames and be filtered as necessary. Eqs. (1) and (2) can also be applied to any ROI separately in which case fₜ(x,y) would represent only the pixels in the ROI. Preferably, the value in Eq. (2) is normalized to the maximum possible value.

Although computationally efficient, block comparison is easily influenced by pixel ‘noise’. Pixel changes between frames arising from non-patient movement will cause false positive movement readings. Pixel noise can be attenuated, but not eliminated by rounding low values to zero or using wavelet transforms [Lee et al 1999]. Simple block comparison is also unable to account for variation in environmental lighting conditions and camera settings. Changes in lighting, for example from drawing the curtain around the patient bed, can result in changes in pixel values that are not due to motion.

To address these issues, a further embodiment (not shown) of the present invention utilizes correlation coefficients to perform motion determination. A normalized correlation coefficient can be used to measure the change between frames of a given image, or ROI within the image.

A frame-to-frame correlation is made for the entire patient area (i.e. the sum of all the patient ROI) and the nursing ROI edge regions (8-11) of the captured image frame (1) using a normalized level of motion in both the patient and nurse areas. The correlation coefficient, rₖ, for each of these (k) regions (i.e. the patient ROI and the edge regions) is defined as the ratio of the covariance between frames over the combined variance of frame t and t+1.

$${r}_{k} ( t + 1 ) = \frac{COV ( {f}_{t} , {f}_{t} + 1 )}{\sqrt{var ( {f}_{t} ) \times var ( {f}_{t} )}} \qquad ( 3 )$$

where “var” is the variance and “cov” is the covariance for the image frames, which can be expanded to define the correlation coefficient as:

$${r}_{k} ( t + 1 ) = \frac{{\sum }_{xy} \{ ( {f}_{t} ( x , y ) = {\overline{f}}_{t} ) ( {f}_{t} + 1 ( x , y ) - {\overline{f}}_{t} + 1 ) \}}{\sqrt{\{ {\sum }_{xy} {( {f}_{t} ( x , y ) - {\overline{f}}_{t} )}^{2} \} \times \{ {\sum }_{xy} {( {f}_{t} + 1 ( x , y ) - {\overline{f}}_{t} + 1 )}^{2} \}}} \qquad ( 4 )$$

where fₜ(x, y) is the pixel value (between 0-255) at location (x,y) at time, t, and \overline{f}ₜ is the average pixel value over the entire region, k, with corresponding definitions for time t+1. The numerator is the covariance between frames for that region and the denominator is the combined variance. Note that the image frames are defined in Equations (3) and (4) for the given region, k.

Equation (4) presents a direct, normalized measure of the change between frames, presenting a clear measure of the level of motion. Therefore, bias due to changes in lighting or differences in camera distance or position that can influence the block comparison method is eliminated. The magnitude of rₖ(t+1) approaches 0 when there is excessive motion because the covariance between frames is very low, and conversely approaches 1.0 when there is little motion. It will be appreciated that this value can be determined (according to the definition of k) for the entire patient regions and nurse areas or combined over selected ROI.

Mathematically, the value of rₖ in Eq. (4) can vary between −1 and +1, depending on the change in motion. However, the magnitude of the motion is typically measured by the variance between frames, and thus represented by the coefficient of determination, Rₖ=r²ₖ, over the range from 0 to +1, eliminating the phase shift information in the sign. As a result, a motion-related agitation index can be defined:
Aₖ(t+1)=1−rₖ(t+1)²=1−Rₖ(t+1) (5)
where k is defined for the nursing edge region ROI (8-11), and/or specified patient ROI. Therefore, Aₖ approaches 0 when Rₖ approaches 1 and the motion is very low between frames. Similarly, Aₖ approaches 1 during extensive motion.

Using Eqs. (4) and (5), different combinations of correlation values for the patient and nurse areas can be measured in real-time. It will be noted that these equations relate to all detected movement, not all of which is agitation related. For instance, low patient motion ROI value and high nursing motion ROI value might indicate the nurses restraining the patient in severe agitation. In contrast, the reversed values might indicate the nurse performing a task that is seen in both the patient and nurse areas with no patient agitation present. Hence, greater patient motion may be the patient, the nurse, or both, each of which represents a different situation. This lack of explicit or crisp dynamics makes this quantification problem suitable for the application of fuzzy logic, where the inputs are the patient and nurse-related agitation indices (0, 1) in Equation (5).

Fuzzy mathematics is an apt tool for classification and diagnostics problems where the dynamics of the system are not well known. Fuzzy system models rely on rules defined from logic built from observational data, rather than sharp formulas, to approximate the unknown dynamic behavior. In this case, the dynamics are defined to range between 0 and 1 as a convenient decimal percentage. The result is a fixed neural network model that is derived from the fuzzy mathematics and rules defined, providing a measure of probabilistic likelihood of each membership function of a fuzzy set representing the likelihood of different levels of agitation (e.g. low, medium, high).

This neural network is not trained and is thus not a neural network in the traditional sense, but rather a means of computationally expressing the rules and fuzzy mathematics. The FIS employs rules and time periods based on known medical treatment protocols and experience to define membership functions (MF) and rules. This known process is called “fuzzification” where crisp, continuous data values are transformed to a discrete, fuzzy (e.g. low-medium-high) classification to be processed by the rules defined to quantify agitation.

The motion agitation index is derived directly from the video frame-to-frame correlation coefficients Rₖ for the nurse and patient ROI motions. Fuzzy logic is used to determine a single motion-related agitation index from these two motions. The patient ROI fuzzy set membership functions (MFs) are shown in FIG. 2 where the x-axis is the patient input motion coefficient (12) from equation (5) and the y-axis is agitation level (13). The same sets are used to represent nurse ROI motion.

FIG. 2 shows the low (14), medium (15), and high (16) MFs. It can be seen that the MFs are not spread regularly along the signal input motion range because motion is measured via the correlation coefficients. This coefficient is equal to 1 if two consecutive frames are identical, however large motions from either the patient or the nurse will never decrease the correlation coefficient to exactly 0 because they do not cover the whole area and the area corresponding to the patient's bed or background will remain unchanged. As a result the membership functions, which estimate low-medium-high agitation levels based on the correlation coefficient input are skewed towards zero. The thresholds set for these MF definitions are obtained empirically from simulated critical care patient motion trials.

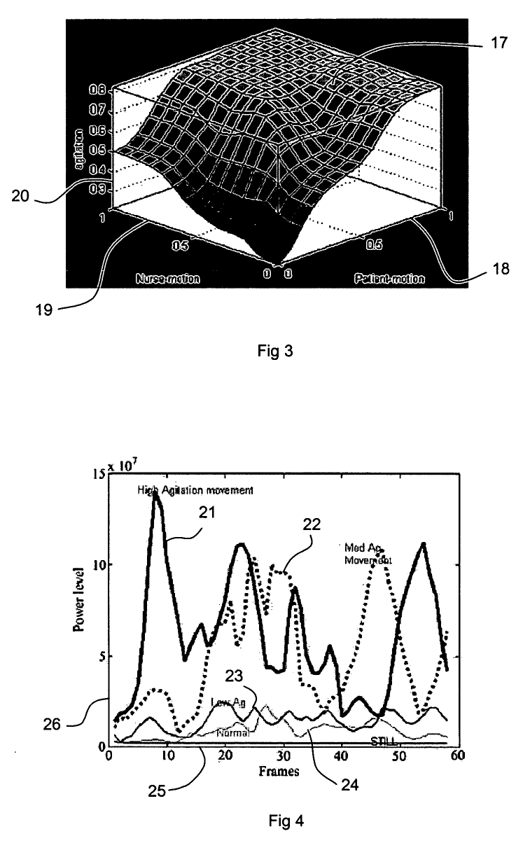

Fuzzy rules are defined to quantify an agitation index value from the MF definitions for both nursing and patient motion and are listed in table 1 below. They determine, using fuzzy mathematics [Terano et al 1992; Kandel 1986], the likelihood that patient agitation is low, medium, or high using the two inputs (patient and nursing motion) and MFs defined. The results of the rules in Table 1 may be represented in the fuzzy transfer surface (17) shown in FIG. 3 that relates the two inputs of patient motion (18) and nursing motion (19) together with the agitation index output (20).

| Rule | Patient-motion | Nurse-motion | agitation |
|---|---|---|---|
| 1. | low | low | low |
| 2. | medium | low | medium |
| 3. | high | low | high |
| 4. | low | medium | low |
| 5. | medium | medium | high |
| 6. | high | medium | high |
| 7. | low | high | medium |
| 8. | medium | high | high |
| 9. | high | high | high |

The final (0, 1) agitation index in FIG. 4 output results from combining the fuzzy (low-medium-high) values and weights to return a crisp number in a known process called “defuzzification”. This process is performed using fuzzy mathematics based on the rules and MFs defined in an inverse manner. Hence, FIG. 3 shows the transfer function of the entire fuzzy logic inference system from crisp input values to crisp output values, in between which the dynamics are defined by the rules and MFs. It will be appreciated however, that alternative rules and refinements to the above rules are possible without departing from the scope of the invention.

The fuzzy logic rules and MFs were defined based on trials using simulated critical care patient agitation videos developed using volunteer actors. These simulated motions mimicked different levels of observed patient agitation, based on inputs from medical staff, and FIG. 1 shows a typical frame (1). The monitoring system was then tested on five critical care patients to prove the initial concept and overall approach. All trials were performed in the Christchurch Hospital Department of Intensive Care, with ethics approval from the Canterbury Ethics Committee. Patient consent was obtained either from the patient or immediate family member.

All critical care patients were receiving fixed concentration morphine (1 mg/mL) and Midazolam (0.5 mg/mL) solution to provide pain-relief and induce sedation. These patients were being weaned from sedation, prior to extubation, to best ensure that a range of agitation might occur. Patients with neuro-muscular blockade, head injury or morbidity were excluded from the tests. Agitation, as assessed by nursing staff, was recorded periodically using a modified Riker SAS with a scale of 0 (calm) to 3 (extremely agitated) [Shaw et al 2003; Shaw et al 2003]. The regular Riker SAS [Riker et al 1999] uses the values 4-7 for this range, with 1-3 representing levels of sedation. The modified scale is more intuitive as it separates sedation and agitation scores, as only agitation levels were assessed.

The initial system was developed and tested using volunteer actors to obtain the transfer function in FIG. 3 before ICU testing. FIG. 4 shows the non-normalised power levels for high (21), medium (22), low (23), and (non-agitated) (24) normal levels of simulated motion. It also shows a motionless response with the expected zero result. The x-axis represents captured video image frames, at 5 fps, and the y-axis represents non-normalised power level (26), as defined by equation (2) and summed over all of the ROI defined in FIG. 1. As expected, the more significant the agitation the greater the power of the motion, which also matches the primary means of agitation assessment [Weinert et al 2001; Foster et a 2001].

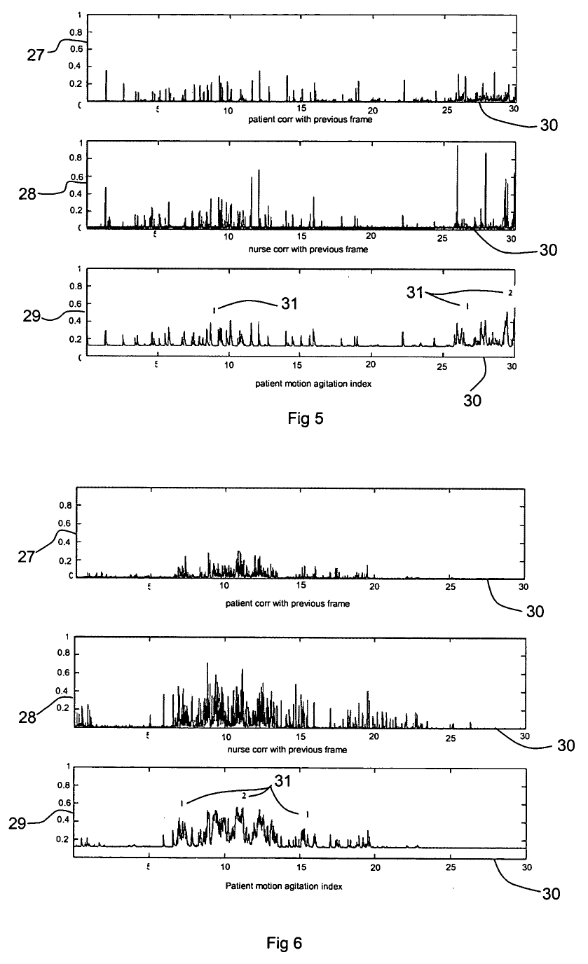

FIGS. 5-7 show the results for three individual ICU patients whose agitation response across patients, as assessed by nursing staff, spanned the range from light to extreme agitation. The y-axis of the first two frames represents the normalised patient motion (27) and nursing motions (28) from 0 to 1, respectively. The y-axis of the third frame is the resulting patient agitation index (29) in the range from 0 to 1. The x-axis (30) represents minutes for each image frame. The large figures in the third frame are nursing assessments (31) of agitation using the modified Riker SAS, which were recorded using the data acquisition software designed for this trial. Each of FIGS. 5-7 represents a small portion of the entire patient survey (which typically extended for 10-20 hours), where significant agitation was manifested and the nursing staff had the time to record Riker SAS assessed agitation.

FIG. 5 shows light level 1 agitation as assessed using the modified Riker SAS. Note that there are intervening periods of low measured agitation using the methods developed and the lack of nursing assessment here is assumed to be level 0 agitation, which does not require nursing attention. Note that the assessed agitation score is beginning to rise at the end of the 30 min sample as evidenced also by increasing amounts of motion for both patient and nurse, ending with the nurse restraining the patient.

The results for Patient 2 are shown in FIG. 6 with a higher and more consistent period of assessed agitation. In this case the patient was calm and then began to manifest a growing level of patient agitation around 7 minutes. This agitation was treated with some additional sedation and a calm state restored after 13 minutes. Note that the greater nursing motion around 20-25 minutes is due to the nursing staff checking on the patient and performing other tasks. Hence, higher nurse motion and low patient motion are appropriately differentiated by the FIS in this case.

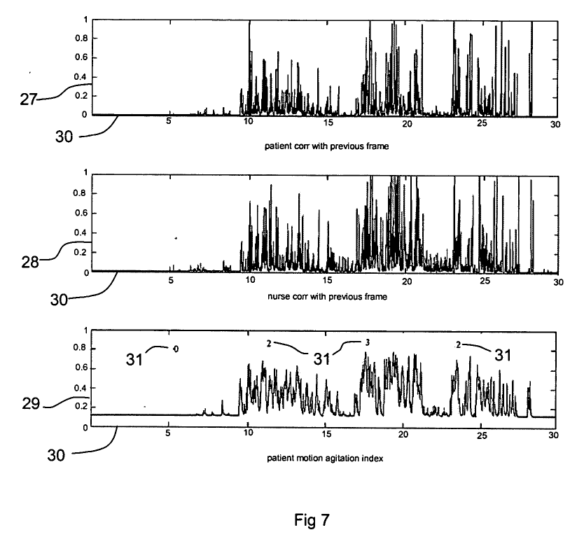

The results for Patient 3 are shown in FIG. 7. This patient experiences a significant and extended bout of severe agitation at about 10 minutes that requires a great deal of restraint as noted by the excessive correlation coefficients for both patient and nursing staff. The assessed agitation levels reflect this severity in the assessments of levels 2-3 by the staff over this 20 minute period. FIG. 7 also shows the very rapid rise in agitation from a calm state, assessed at 0, over the first 10 minutes, illustrating the rapid changes that can occur in these patients.

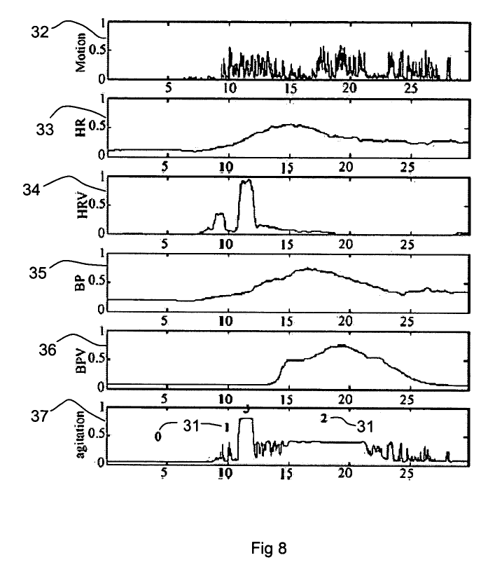

FIG. 8 shows the agitation level assessed using patient motion (32), as well as for assessments made using physiological based metrics for heart rate (33), systolic blood pressure (34), heart rate variability (35), and blood pressure variability (36). The readings were obtained from the same patient as FIG. 8, but over a separate time period where all three levels of Riker SAS agitation were observed. In this case agitation levels assessed using the above cardiovascular metrics (33-36) were performed using the method described more fully herein below. The final frame shows the combined agitation levels (37) determined from the fuzzy mathematical combination of MFs for each metric before the output of the final crisp patient agitation value.

As a result, a given metric is only dominant in the final frame when all the others are low and/or falling. It can be seen also that the motion assessed agitation value appears to correlate well with the physiological measurement based metrics (33-36), which have also been independently shown to correlate with subjective nursing staff assessments in proof of concept studies [Shaw et al 2003; Lam et al 1983]. These results indicate that both physiological and patient motion approaches to quantifying agitation, based on correlation with nursing staff assessment, also match.

FIG. 4 illustrates that the increased motion seen in simulation of various levels of ICU patient agitation has power that can be directly correlated to patient agitation and used to develop rules about how to quantify that level of agitation. FIGS. 5-8 show the results for three ICU patients that manifested differing levels of patient agitation spanning the 0-3 range of the modified Riker SAS scale. In each case, increasing levels of patient motion resulted in an increased agitation index. These increases correlated consistently and well with nursing assessed agitation levels. In addition, calm periods, where nurses noted no agitation and made no assessment, were not recorded as false positive results. As patient motion is often the primary input used in subjective assessments of patient agitation [Weinert et a/2001; Foster et a/2001], good correlation indicates the effectiveness of the fuzzy system in differentiating between motion from nursing staff and patient care, and patient agitation.

Hence, the short list of fuzzy inference system rules and membership functions developed is effective in enabling this direct correlation between subjective and computer based assessment of the patient motion signal.

FIG. 8 represents the correlation between physiologically quantified agitation using four different physiological signals [Shaw et al 2003; Lam et al 1983] and agitation quantified based on the motion sensing approach presented. Motion-based agitation sensing is more directly correlated to the effective signals used in subjective nursing staff assessments of patient agitation [Weinert et al 2001; Foster et al 2001] so their correlation is not surprising.

However, the physiological measurements are based on the hypothesis that agitated motion and agitation itself are manifested in the autonomic nervous system responses seen in these physiological signals, and is explored further below. These physiological signals also show good correlation. However, the combination of all of these metrics is seen to correlate equally well, if not better, than the patient movement metric alone, illustrating the potential for such a multi-signal approach.

Patients 1 and 3 show similar levels of nursing and patient motion in FIGS. 5 and 7, whereas Patient 2 has greater nurse motion than patient motion in FIG. 6. However, in each case the fuzzy quantifier is selectively judging the contributions of each. One major confounding issue is that nursing staff may be more or less involved with the patient depending on the level of agitation so that high levels of agitation may see lower relative levels of nursing motion for safety reasons than lower levels. A second is that each nurse treats aggravated motion differently, which leads to different levels of nursing motion, relative to patient motion, for the same agitation level. The fuzzy logic rules have shown the basic capability of distinguishing nursing and patient motion for judging agitation.

It will also be appreciated that the results for patient motion in FIGS. 5-8 are all non-smooth due to the frame-to-frame definitions of the correlation coefficient and agitation index values in equations (4) and (5). The noisy appearance of these signals is distinctly pronounced for the low agitation levels seen in FIG. 5. Clinically, smoother responses would be more suitable as a feedback signal for controlling sedation administration. The data presented in these FIGS would be smoother if a higher frame rate or additional filtering for RMS or moving average values were used. The use of longer time period windows, rather than immediate frame-to-frame calculations, would also smooth these results.

The present invention provides a method of physiologically quantifying patient agitation based on reliable, objective digital imaging-based motion sensing. The concept quantifies patient and nursing staff motions and uses a fuzzy inference system with simple rules to differentiate between motion due to patient care and manifestations of patient agitation to provide an objective, continuous measurement of agitation. The basic method splits the image into patient and nursing (edge) ROI and determines a normalized measure of motion power in each. The method can also be extended to individually examine motion of specific body parts or areas of the patient.

Results show that agitation can be assessed in sedated ICU patients and quantified using this approach, including differentiating periods of calm. Periods of detected agitation in ICU patients correlate well with subjective assessment by trained medical staff using the modified Riker SAS. These results show that agitation can be quantitatively measured and assessed using this computationally inexpensive digital imaging approach. Further results show the method correlates well with agitation assessed using physiological signals and that they can be combined into a final agitation metric with good correlation for the subject tested. Clinically, this research presents a system capable of providing real-time assessment of patient agitation where nursing staff are not always unbiased or available. These measurements facilitate a better understanding of patient agitation as well as being used to guide sedation administration and selection.

As discussed above, agitation may also be assayed from monitoring physiological metrics including cardiovascular and respiratory as well as patient motion. The hypothesis behind this approach is that patient agitation can be measured by determining the amount of sympathetic nervous system activity present in readily available biomedical signals such as HRV, BP, BPV, respiratory rate (RR) heart rate derivative (HRD); blood pressure derivative (BPD); temperature; cardiovascular metrics (including cardiac output (CO), diastolic blood pressure, cardiac filling volumes); EEG/brain wave measurements and the like. As a patient manifests agitation, sympathetic response to this stress and resultant motion leads to changes in these physiological signals. More specifically, as agitation manifests, heart rate and blood pressure have been observed to rise. These increases lead to decreased HRV, and elevated BP and BPV levels (Pfister et al 2001).

Therefore, an aim of the present invention is to measure these physiological signals and determine to what level and in what manner they correlate with the assessed agitation, enabling a consistent, quantifiable measure of patient agitation to be created for each signal.

Thus, in a further preferred embodiment, HRV and BPV are measured by examining the variation in the power spectral density (PSD) of heart rate and blood pressure respectively. HRV examines the R-R interval between heart beats (tachogram), and BPV examines the changes in systolic blood pressure (systogram). The basic signal processing steps for quantifying agitation include (1) peak detection and interval calculation, (2) spectral estimation and calculation of power in different frequency bands and (3) determination of patient agitation from changes in signal dynamics.

Considering these stages individually;

Peak Detection:

Peak detection is similar to both heart rate and systolic BP and is consequently discussed for heart rate only for the sake of conciseness. The QRS complex in the ECG signal can be easily detected using a Haar wavelet (Lee et al 1999). The continuous wavelet transform (WT) coefficients determined using the Haar wavelet are located at the same times as the QRS peaks in the ECG, enabling detection of the QRS complex, the occurrence of R-peaks and the calculation of the R-R interval between peaks.

The Haar wavelet is used because of its simplicity, thus providing a fast algorithm necessary for this real-time application and its ability to detect singularities (edges) in the signal. Hence, a simple threshold-based QRS detection algorithm can be applied to the wavelet transform coefficients to find the R-peaks. The same technique is used to identify systolic and diastolic blood pressure values from a real-time blood pressure signal. This technique also eliminates the difficulties with baseline shifts in the measured data as the shape of the Haar wavelet picks out the peak values, and zeroes the remainder of the signal, resulting in consistent, unbiased values at the peak locations (Lee et al 1999).

Spectral Estimation:

As HRV and BPV values must be determined continuously in real time for this application, frequency domain analysis can be used to obtain the power spectrum for the following standard frequency bands: very low (VLF) 0.0033-0.04 Hz, low (LF) 0.07-0.14 Hz, high (HF) 0.150.4 Hz. The power in these frequency bands varies due to the influence of the sympathetic/parasympathetic nervous system responses and can therefore be used for measuring the state of the nervous system (Mainardi et al 1997, McCraty et al 2001), and hence agitation.

Commonly used nonparametric Fourier transform (FT) based spectral estimation methods suffer drawbacks, including loss of time information when transforming the signal to the frequency domain. Thus, it is difficult to immediately tell when a special event occurred. The Fourier basis is therefore ill-suited for non-stationary signals, which are especially important for HRV signal processing and spectral estimation as the (long-term) R-R and HRV signals are highly non-stationary. In addition, HRV follows a circadian rhythm, so the parameters that describe the HRV (variance and mean) will never be completely stationary, particularly for a critical care patient. Thus, a parametric method, such as auto-regression (AR), is more suitable.

Frequency analysis of R-R and systolic blood pressure signals is performed using an adaptive autoregressive (AR) spectral estimation method with 100 initial samples from the tachogram for the HRV and systogram for the BPV. The AR modeled signal is defined (Marple 1987) as

$$x ( n ) = - {\sum }_{k = 1}^{p} {a}_{k} x ( n - k ) + e ( n ) \qquad ( 6 )$$

where p is the model order, aᵢ are the AR model coefficients, x(n−k) are the prior signal samples and e(n) is zero-mean white noise with variance ρ_{w}. Note that equation (6) is in beats and that conversion back to time scales is done by using the average R-R interval value for a given frame.

The PSD, P_{AR}, can be calculated from the following formula:

$${P}_{AR} = T {σ}_{∞} {\vert \frac{1}{A ( f )} \vert}^{2} \qquad ( 7 )$$

where T is the sampling interval used for scaling and |A(f) | is the frequency response obtained from the AR coefficients aᵢ. Using the fast recursive least squares (RLS) algorithm enables an update of the spectral estimation every time a new sample is available (Marple 1987). The AR coefficients are therefore estimated from the signal samples by calculating the forward linear prediction error for the N^{th} sample, defined as

$${e}_{p , N}^{f} ( n ) = x ( n ) + {\sum }_{k = 1}^{p} {a}_{p , N} ( k ) x ( n - k ) . \qquad ( 8 )$$

Minimizing the weighted squared error to sample N yields the forward prediction error

$${p}_{p , N}^{f} = {\sum }_{n - 1} {ω}^{N - n} {\vert {e}_{p , N}^{f} ( n ) \vert}^{2} \qquad ( 9 )$$

where ω is a forgetting factor that determines the importance of past values. The time index update for the forward linear prediction coefficients is defined as
α_{p,N+1}^{f}=α_{p,N}^{f}−e_{p,N}^{f}(N+1)c_{p-1,N} (10)
where c is a gain vector determined from
R_{p,N-1}c_{p,N}=ω⁻¹x*_{N}(N) (11)
where R is the autocorrelation matrix of the input signal estimated from the samples.

The fast RLS algorithm introduces the backward prediction update, which uses vector operations instead of time-consuming matrix calculations, making it effective for real-time applications. A key advantage of this sequential algorithm is its ability to track changes in the signal variance and mean, allowing this method to adapt to the characteristics of the signal in real time. It also eliminates the requirement that the signal be stationary, removing an important limitation with many other methods.

After estimating the PSD, the spectral power in the VLF, LF and HF frequency bands are calculated. The ratios VLF/HF for HRV and HF/VLF for BPV are then used as an input for a fuzzy inference system (FIS). These signals measure the decrease in HRV and increase in BPV, respectively, as agitation manifests. Hence, both ratios are expected to rise when agitation occurs.

Determination of Patient Agitation from Changes in Signal Dynamics:

A previously discussed, fuzzy mathematics is a very appropriate tool for classification and diagnostics problems where the dynamics of the system are not well known. Fuzzy system models rely on rules defined from fundamental logic and/or observational data, rather than sharp formulae to approximate the unknown dynamic behavior. In the present embodiment, the dynamics are defined to range between 0 and 1 as a convenient decimal percentage. It will be appreciated however, that alternative ranges may be employed. The result is a fixed neural network model that is derived from the rules defined and provides a measure of probabilistic likelihood through the definition of membership functions representing the likelihood of different levels of agitation (e.g., low, medium and high). Therefore, this application employs rules and time periods based on known medical treatment protocols and experience to define membership functions and rules. This process utilizes ‘fuzzification’ where crisp data values are transformed to a fuzzy low-medium-high classification to be processed by the rules defined to quantify agitation.

Four FIS inputs were used for each signal: the current signal value (V1) and its mean value over the prior 5, 10 and 20 min (V5, V10, V20). These values were chosen based on clinical expertise and the action time of the sedatives used (3-10 min). Essentially, these time periods represent instantaneous (1), immediate (5), sedative effect time (5 and 10) and long term (20) states of patient agitation. This technique allows changes in the signal to be followed and facilitates the detection of longer-term trends. Nine fuzzy rules (as shown in table 2 below) were developed to define the individual agitations levels for each input signal. In a second step the individual agitation levels, obtained for HRV, systolic blood pressure and BPV, are then combined to create a single agitation value.

TABLE 2
Fuzzy interface system (FIS) rules for a systolic BP analysis.

| Rule | V1 | V5 | V10 | V20 | Agitation |
|---|---|---|---|---|---|
| 1 | Low | — | — | — | Low |
| 2 | Medium | High | — | — | Low |
| 3 | Medium | Medium | Low | Low | Low |
| 4 | Medium | Medium | Medium | Medium | Low |
| 5 | Low | Low | Low | Low | Low |
| 6 | High | High | High | High | High |
| 7 | High | Low | Low | Low | High |
| 8 | High | Medium | Low | Low | High |
| 9 | High | Medium | Medium | Medium | Medium |

Table 2 shows the fuzzy rules used for applying the FIS to the systolic BP values as input signal. Rule 1 looks only at the current value (V1) and if this value is low the resulting agitation level is low, regardless of the other inputs. This rule assumes that agitation is not manifested with a low systolic blood pressure value, matching patient observations. The other rules are based on similar fundamental assumptions based on known observation and protocol, and compare the actual value to prior values and means to detect changes and thus agitation.

The same rules are used for BPV and HRV signal values for this initial research. Finally, the fuzzy value is returned to a crisp (0, 1) value using fuzzy logic just as in the initial fuzzification process, but applied to the outputs of every rule.

The fuzzy rules were defined based on extensive observation of sedated ICU patients, and the experience and protocols of the ICU medical staff at Christchurch Hospital. They represent an attempt to quantify the agitation level from physiological signals.

The clinical trial procedure adopted was as follows: HRV and BPV were first verified as appropriate indicators for agitation induced in normal, healthy subjects before testing the method developed in ICU patients. All tests were performed in the Christchurch Hospital Department of Intensive Care Medicine, with ethics approval obtained from the Canterbury Ethics Board.

For normal subjects, Stroop's color word test (CWT) (Sijska 2002, Freyschuss et al 1988) and a cold pressor (CP) test are known to induce mental stress and pain respectively, and are thus a surrogate of patient agitation. These tests allow reproducible, standardized levels of this type of agitation to be induced, enabling inter-subject comparisons. Tests were performed in the morning, and the test subjects were directed not to drink coffee or tea and to have only a light breakfast on the day of the test. The subjects were tested lying down in a comfortable reclining chair to provide a body position similar to that of an ICU patient.

The CWT consisted of 630 slides automatically presenting one slide every 2 sec. On every slide there was a color name (blue, green, red, or yellow) written in text of a non-corresponding color. Over headphones the subject heard a third color name spoken. The goal was to mark the color the word was written in on a separate paper containing the color words in a random order for every slide. Before and after the CWT test the subject had a 20 min rest period. ECG and blood pressure were obtained from all subjects. For the CP test following the CWT the subject puts their dominant arm into ice cold water for 1 min. A second CP test is performed after a 10 min rest period, and the trial concludes with another 10 min rest period. Heart rate data was recorded using a Marquette monitor and PC for 13 subjects.

Blood pressure data was measured every 3 min using an automated cuff. Due to the long time between blood pressure samples BPV could not be obtained for normal subjects. Therefore, systolic blood pressure was analyzed as a surrogate to determine its correlation with the induced agitation.

After developing and proving the concept for healthy subjects, five ICU patients were monitored using the same system to obtain HRV and BPV to prove the concept and validate the use of the CWT and CP tests as surrogates for patient agitation. All patients were receiving fixed concentration of morphine (1 mg ml-1) and Midazolam (0.5 mg ml-1) solution to provide pain-relief and induce sedation. R-R interval and blood pressure data were sampled at 1000 Hz, twice the typically recommended rate for HRV analysis (Malik 1996), to ensure that each R-peak was accurately captured. Heart rate data was obtained from the Marquette monitors, and arterial blood pressure was measured invasively using an existing arterial catheter. Systolic blood pressure was also analyzed for comparison with the normal subjects, along with BPV from the systogram. All ICU patients tested were being weaned from sedation, prior to extubation, to best ensure that a range of agitation would occur as sedation was removed. In addition, all patients were video recorded to enable review of calm and agitated periods.

Patients with neuro-muscular blockade, head injury or high morbidity were excluded. Agitation, as assessed by nursing staff, was recorded periodically using a modified Riker SAS (shown in table 3) with a scale of 0 (calm) to 3 (extremely agitated) (Shaw et al 2003). The regular Riker SAS (Riker et al 1999) uses the values 4-7 for this range, with 1-3 representing levels of sedation. The modified scale used for this research is more intuitive as only agitation levels are assessed.

TABLE 3
Modified Riker sedation-agitation scale (SAS).

| Sedation/ | |
|---|---|
| agitation score | Description |
| 3 | Dangerous agitation |
| | Pulls at endotracheal tube (ET), tries to remove |
| | catheters, climbs over bedrail, strikes staff, thrashes |
| | from side to side |
| 2 | Very agitated |
| | Does not calm, despite frequent verbal reminding |
| | of limits, requires physical constraints, bites ET |
| | tube |
| 1 | Agitated |
| | Anxious or mildly agitated, attempts to sit up, |
| | calms down to verbal instructions |
| 0 | Calm and cooperative |
| | Calms, awakens easily, follows commands |
| −1 | Sedated |
| | Difficult to arouse, awakens to verbal stimuli or |
| | gentle shaking but drifts off again, follows simple |
| | commands |
| −2 | Very sedated |
| | Arouses to physical stimuli but does not |
| | communicate or follow commands, may move |
| | spontaneously |
| −3 | Unarousable |
| | Minimal or no response to noxious stimuli, does |
| | not communicate or follow commands |

During the trial the nursing staff were encouraged to input their agitation score as regularly as other duties allowed. No interruption or alternation to any of the existing methods by which agitation is currently assessed or sedation administered. The survey data is thus strictly observational in this respect. The duration of any score is only effective until changed from outside input or growth of agitation. It should be noted that 0 values are not recorded but that video record shows no patient motion or agitation during these periods. This feature is deliberately introduced to avoid an excessive number of interruptions to normal duties in a working ICU.

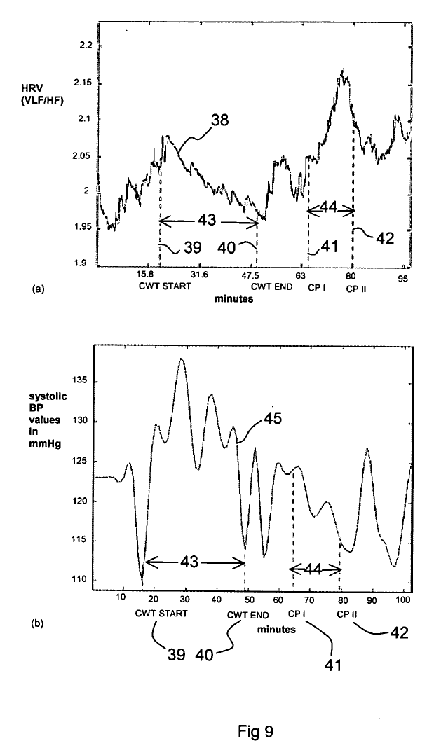

FIG. 9(a) shows the HRV signal (38) obtained from a typical normal subject with the start times (39, 41) and end times (40, 42) respectively for the CWT (43) and CP (44) tests. It can be seen that both tests cause an increase in the HRV signal (38) (given by the ratio VLF/HF) as the HF component drops when the variability in heart rate decreases during the surrogate of patient agitation induced. The increase during the CWT (43) is smaller than the increase during CP (44) tests. It can also be seen that the VLF/HF ratio (38) decreases during the second half of the CWT test (43), indicating a gradual habituation. The ratio stays relatively high after the second CP test (44), showing the long time required to recover.

FIG. 9(b) shows the systolic blood pressure values (45) for the same subject. The reaction to the CWT (43) is much stronger compared to the relative change in HRV (38) in FIG. 1(a). The inverse result is obtained during the two CP (44) tests. Hence, it can be seen the subject's reaction to mental stress and pain is different for HRV (38) and systolic blood pressure (45).

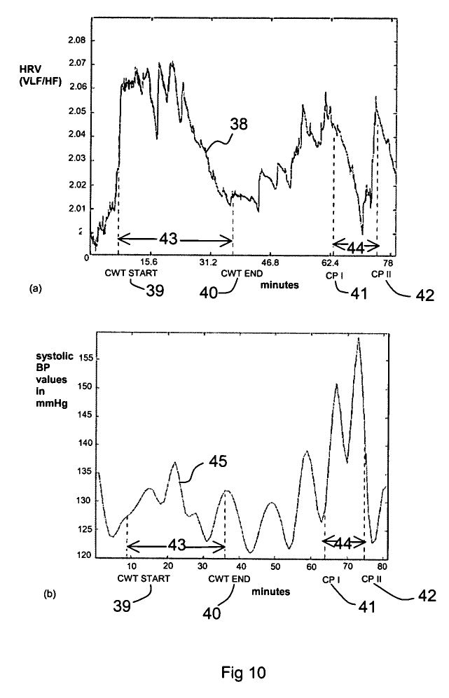

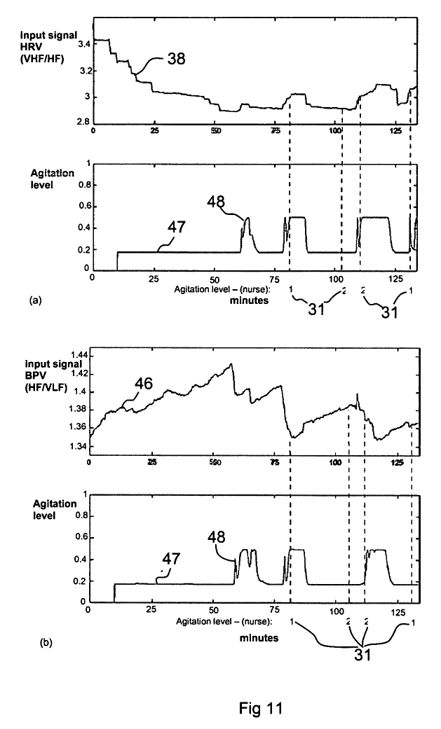

FIG. 10 shows the same results for a second normal subject, where the HRV (38) results are different to those in FIG. 9(a). The VLF/HF ratio increases strongly during the CWT (43) and even a few minutes before it begins, which is attributed to anticipatory nervousness. However, it can be seen that the ratio decreases during periods of calm as with the subject in FIG. 9(a).

FIG. 10 (b) shows systolic blood pressure (45) results that are similar to those in FIG. 9(b) with rises during agitation and falling values in calm periods. This subject's HRV (38) signal appears to react more strongly to mental stress than physical pain, in contrast to the blood pressure that shows greater change during the two CP (44) tests.

The results in FIGS. 9 and 10 for two normal subjects show both commonality and variability of physiological response to agitation between very similar subjects, highlighting the need to use several biomedical signals rather than relying on just one. Similar results were obtained for the 11 other normal subjects with slight differences in the individual reactions to pain and mental stress. In general, the results show an increase during the CWT (43) and CP (44) tests and a decrease during calm periods. Hence, both HRV (38) and systolic blood pressure (45), and by analogy BPV, can be reasonably correlated with the surrogate of agitation induced in normal subjects.

FIGS. 11(a) and 11 (b) shows the VLF/HF (HRV) (38) and HF/VLF (BPV) (46) ratios used as the input for the fuzzy inference system (FIS) and the individual agitation level (47) outputs that result for a typical ICU patient. The detected patient agitation correlates very well with the agitation levels provided by trained nursing staff using the Riker SAS scale (31) on the 0-3 scale as shown below the x-axis of the figures. FIGS. 11(a) and (b) also both show a potential lower agitation period (47), which was undetected by the nurses but was seen in the video record on review. Note that in FIG. 11(b) the quantified agitation level resulting from the fuzzy rules rises slowly, or late. Despite this lack of FIS optimization, the results do correlate well enough with traditional subjective assessment methods to provide a workable system.

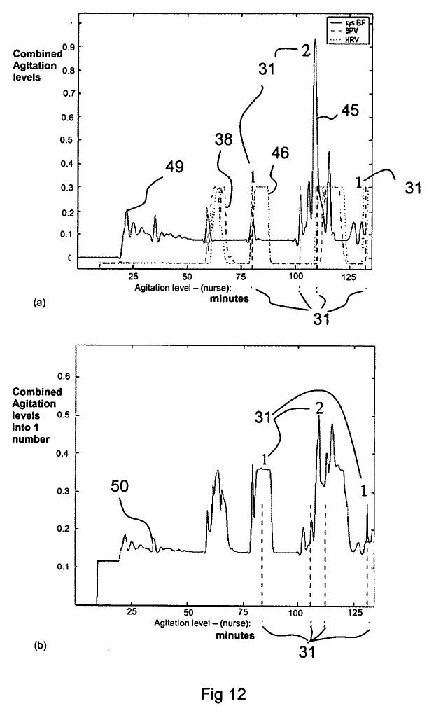

FIG. 12(a) shows the combination of the individual agitation levels (49) for three different input signals, HRV (38), BPV (46) and systolic BP (45) for the same ICU patient, with salient agitation assessments also provided in the plot itself. These individual signals are then combined into a single overall agitation number (50) by calculating the mean agitation level, as shown in FIG. 12(b). Currently, the systolic BP values are dominant, which will be taken into account for further development of the algorithms. The first 20 min of testing does not produce an agitation level as this time period is used for initializing the FIS signals.

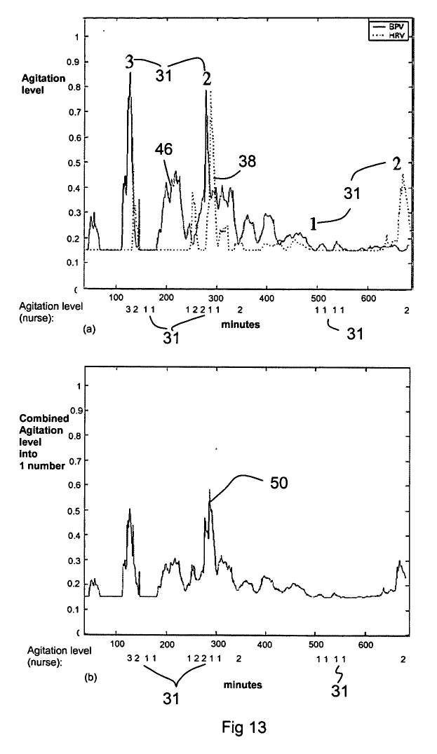

FIG. 13(a) shows the agitation level for a second ICU patient over 700 min for HRV (38) (dotted line) and BPV (46) (solid line) only. It also shows the agitation level as evaluated by nursing staff with salient values (31) also in the plot itself. This specific patient experienced both more frequent and greater agitation during the monitored period than the patient in FIGS. 11 and 12, as seen in the assessed agitation along the x-axis. Both HRV (38) and BPV (46) are able to identify periods of agitation. They also show the general relative grade of agitation. FIG. 13(b) shows the overall agitation number (50) obtained by calculating the mean of the agitation levels determined from the two input signals, HRV (38) and BPV (46).

Similar results were obtained for the other tested ICU patients with slight differences in the individual reactions during periods of agitation. In general, the results show an increase during nurse assessed agitated periods and a decrease during calm periods. Hence, HRV (38) BPV (46), as well as systolic blood pressure (45), are reasonably correlated with the agitation encountered in ICU patients as graded by nursing staff using the modified Riker SAS scale (31).

Furthermore, the results are consistent when comparing normal subjects to ICU patients. This result indicates a good correlation between the surrogate of agitation induced in normal subjects and the agitation found in ICU patients. The first consequence is that further developments may be initially trialed on normal subjects with reasonable confidence of the results transferring to ICU patients. Secondly, this similarity also provides initial insight into potential mechanisms of agitation in sedated ICU patients.

The adaptive autoregressive analysis applied to HRV and BPV is shown to detect changes in normal subjects during periods of agitation when compared to periods of calm. In addition, increases in systolic BP also correlate well with agitation, as decreases do with calm periods. When compared to ICU patients graded to be agitated under the Riker SAS scale by trained nurses, AR analysis and the initial FIS rules developed show good correlation. For HRV, the VLF/HF ratio is shown to increase during agitation and decrease during calm periods. Research indicates that the HF component is due to parasympathetic activity and VLF due to sympathetic activity, so this result is consistent with the reported data (McCraty et al 2001). In addition, Riker SAS scale graded calm periods coincide with calm readings on the agitation scale.

Together with a fuzzy logic quantifying process, the change in PSD can be quantified into a single number. The resulting agitation level is between 0 and 1, with 0 representing a non-agitated state and 1 representing a fully agitated state. For ICU patients, the results correspond well with periods of agitation as graded by nurses.

It should be noted that the comparison of medical staff assessed agitation, using the Riker SAS, and the physiological monitoring performed by the present invention are measuring different metrics. The physiological monitoring/assessment method developed measures changes in HRV and BPV as surrogates for agitation, based on testing of normal subjects. In contrast, the subjective methods such as the Riker SAS are based primarily on medical staff assessments of undesirable patient motion. Hence, these approaches represent two different definitions of agitation, with the latter being used to guide sedation administration. The earlier-described quantitative monitoring of patient motion provides a yet further means of validation of the ability of physiological signals and signal processing to provide a consistent surrogate measure of agitation.

Naturally, all systems have potential limitations. There is a large body of literature that discusses the suppression, or other impact, of a variety of conditions, such as sepsis, drug effects and cardiovascular disease, that could cause difficulties with this measurement approach. This supports the use of multiple measures, rather than a single signal. More specifically, as seen most clearly in FIG. 13, there are times when the HRV (38) responds and times when BPV (46) responds to match assessed agitation, or sometimes both.

In conclusion, the method of physiologically quantifying patient agitation presented is based on reliable, objective physiological signals. The present invention is capable of quantifying autonomic nervous system interactions to provide an objective measurement of agitation. Adaptive autoregressive (AR) signal processing techniques are used to analyze heart rate (HRV) and blood pressure (BPV) variability and are combined with a fuzzy quantifier to measure agitation levels.

Results show that agitation in normal subjects can be assessed and quantified using this approach, including differentiating periods of calm. Additionally, it has been shown that detected periods of agitation in ICU patients correlate well with subjective assessment by trained medical staff using the modified Riker SAS and with the objective assaying of patient motion described in the earlier embodiment. These results show that agitation can be quantitatively measured and assessed using common biomedical signals. Finally, agitation induced in normal subjects correlates well to agitation in ICU patients, as both show similar changes in the measured biomedical signals during agitated periods. This result will prove useful for further research and development of the method, as normal subjects can be used prior to further clinical testing.

It will be appreciated that the present invention may be further applied to wider medical and non-medical applications, including controlling the administration of sedatives according to the quantified agitation determined by the above methods. The ability to provide accurate sedative amounts without patient discomfort and nursing staff risk of under-sedation and the expense and prolonged patient recovery of over-sedation provides significant advantages. The quantified agitation monitoring system of the present invention may be linked directly to any convenient automated sedative administration system (not shown) for oral or infusion sedative administration.

In a further embodiment, the quantified agitation may also be used to provide an alarm system for use in non-ICU clinical locations where patients typically receive reduced nursing monitoring. In such applications, the system may provide an alert to nursing staff if the patient agitation exceeds predetermined threshold values. Consequently, nursing staff may monitor an increasing number of patients for agitation with a reduced need for frequent physical observations.

In non-medical applications, the sensing technology of the present invention may also be used to detect abnormal or significant user motions. Applications include monitoring for user fatigue during driving, flying or such activities, where the user may become drowsy, inattentive or even fall asleep. The motion detection aspects of the present invention may be used to trigger an alarm if the user ceases any movement below a predetermined threshold level.

In a converse application, individuals under stress such as during police questioning or the like may exhibit involuntary displacement gestures, i.e. agitation, which currently is not quantified to any degree. As an aid to a policeman's visual observations, the present invention may be utilized to provide a quantitative measurement of such gestures to facilitate analysis of the subject's voracity.

### REFERENCES

Bianchi A, Mainardi L, Meloni C, Chierchia S and Cerutti S 1997 Continuous monitoring of the sympatho-vagal See endnote 1 balance through spectral analysis IEEE Eng. Med. Biol.
Carrasco G 2000 Instruments for monitoring intensive care unit sedation Crit. Care 4 217-25
Cohen I L 2002 Management of the agitated intensive care unit patient Crit. Care Med. 30 S97-123
J. G. W. Foster, S. K. Kish, C. H. Keenan, Critical care practice and procedures: a national survey of critical care nurses' practices relating to administration of neuromuscular blocking agents, Am. J. Critic. Care 10 (3) (2001) 139-145.
Fraser G L and Riker R R 2001 Advances and controversies in sedating the adult critically ill NY Health-Syst. Pharm. 20 17-24
Freyschuss U, Hjemdahl P, Juhlin-Dannfelt A and Linde B 1988 Cardio-vascular and sympatho-adrenal responses to mental stress: influence of β-blockade Am. Physiol. Soc. 158 1443-51
Jacobi J 2002 Clinical practice guidelines for the sustained use of sedatives and analgesics in the critically ill adult Crit. Care Med. 30 119-41
Kandel A 1986 Fuzzy Mathematical Techniques with Applications (Reading, Mass.: Addison-Wesley)
Kress J P, Pohlman A S, O'Connor M F and Hall J B 2000 Daily interruption of sedative infusions in critically ill patients undergoing mechanical ventilation N. Engl. J. Med. 342 1471-7
Z. Lam, C. Starfinger, J. G. Chase, G. M. Shaw, F. Agogue, Movement Quantification for Use in Agitation Quantification in Sedated ICU Patients, World Congress on Medical Physics and Biomedical Engineering (WC2003), Sydney, Australia, 24-29 Aug. 2003, CD-ROM, ISSN: 1727-1983, 4 pp.
Lee S W and Nam B H 1999 Peak detection of ECG signal using wavelet transform and radial basis functions SICE pp 1067-70
J. de Lemos, M. Tweeddale, D. Chittock, Measuring quality of sedation in adult mechanically ventilated critically ill patients, The Vancouver Interaction and Calmness Scale Focus Group, J. Clin. Epidemiol. 53 (2000) 908-919.
Lombardi F, Sandrone G, Pernpruner S, Sala R, Garimoldi M, Cerutti S, Baselli G, Pagani M and Malliani A 1987 Heart rate variability as an index of sympatho-vagal interaction after acute myocardial infarction Am. J. Cardiol. 60 1239-45
Mainardi L T, Yli-Hankala A, Korhonen I, Signorini M G, Bianchi A M, Takala J, Nieminen K and Cerutti S 1997 Monitoring the autonomic nervous system in the ICU through cardiovascular variability signals IEEE Eng. Med. Biol. 16 64-75
Malik M 1996 Heart rate variability-standards of measurement, physiological interpretation, and clinical use Eur. Heart J. 17 354-81
Marple S L Jr 1987 Digital Spectral Analysis: With Applications (Englewood Cliffs, N.J.: Prentice Hall)
McCraty R, Atkinson M and Tomasino D 2001 Science of the heart: research overview and summaries Institute of Heart Math, Publication 01-001
Pfister E A, Rüdiger H and Scheuch K 2001 Leitlinien-herzrhythmusanalyse in der arbeitsmedizin Deutsche Gesellschaft fur Arbeitsmedizin und Umweltmedizin e.V AWMF-Leitlinien-Register 002(020)
Riker R R, Picard J T and Fraser G L 1999 Prospective evaluation of the sedation-agitation scale for adult critically ill patients Crit. Care. Med. 27 1325-9
Sessler C, Gosnell M, Grap M, Brophy G, O'Neal P, Keane K, Tesoro E and Elswick R 2002 The Richmond agitation sedation scale: validity and reliability in adult intensive care unit patients Am. J. Respir. Crit. Care Med. 166 1338-44
Shaw G M, Chase J G, Rudge A D, Starfinger C, Lam Z, Lee D, Wake G C, Greenfield K and Dove R 2003 Rethinking sedation and agitation management in critical illness Crit. Care Resuscitation 5 109-20
G. M. Shaw, R. A. Dove, K. M. Greenfield, A. D. Rudge, J. G. Chase, A Computerised Approach to Sedation Administration in Critically III Patients, in: Proceedings of the ANZICS ASM on Intensive Care, Dunedin, New Zealand, 4-7 Mar. 2003.
Sijska E 2002 The Stroop colour-word test in psychology and biomedicine Acta Univ. Polacki. Olomuc., Gymn. 32 See endnote 2
Szokol J W and Vender J S 2001 Anxiety, delirium, and pain in the intensive care unit Crit. Care Clin. 17
Terano T, Kiyoji A and Sugeno M 1992 Fuzzy Systems Theory and Its Applications (San Diego, Calif.: Academic)
Weinert C, Chian L and Gross C 2001 Sedating critically ill patients: factors affecting nurses' delivery of sedative therapy Crit. Care 10 156-67
Wiener-Kronish J P 2001 Problems with sedation and analgesia in the ICU Pulm. Perspect. 18

1. A method of objectively assaying agitation in an individual subject or patient, said method including;
automated monitoring of at least one metric of;
a. a patient's autonomic nervous system (ANS);
b. expert systems or rules delineating other clinical events from agitation and/or
c. physical movement of one or more defined region(s) of interest (ROI) of the patient's body,
performing signal processing on physiological signals associated with the monitored metric and
quantifying agitation from changes in said processed physiological signals.

2. The method as claimed in claim 1, wherein said quantifying agitation step provides a corresponding agitation value within a defined agitation index.

3. The method as claimed in claim 1, wherein said physiological signals include;
heart rate variability (HRV);
blood pressure (BP);
blood pressure variability (BPV);
respiratory rate (RR);
heart rate derivative (HRD);
blood pressure derivative (BPD);
temperature;
cardiovascular metrics, including cardiac output (CO), diastolic blood pressure, cardiac filling volumes;
EEG/brain wave measurements;
physical movement of one or more defined regions of interest (ROI) of the individual's body.

4. The method as claimed in claim 1, wherein an automated monitoring of at least one metric associated with physical movement of one or more defined regions of interest (ROI) of the subject's body, and comprises the steps:
image capture of at least one ROI;
determination of motion in a ROI;
quantification of relative subject agitation.

5. The method as claimed in claim 4, wherein said determination of motion in ROI step further includes the:
determination of power spectral density (PSD).

6. The method as claimed in claim 4, wherein said method includes the further step of
calculating a corresponding agitation value within a defined agitation index using a fuzzy logic inference system.

7. The method as claimed in claim 1, wherein the subject's body is subdivided into defined regions of interest (ROI) according to the primary body portions likely to exhibit movement.

8. The method as claimed in claim 7, wherein the subject's ROI include the subject's limbs and head for a supine bedded subject.

9. The method as claimed in claim 4, wherein said determination of motion distinguishes between a subject's motions and third party individuals.

10. The method as claimed in claim 9, wherein said third parties may include nursing of medical staff, patient relatives.

11. The method as claimed in claim 1, wherein, said at least one third party ROI is provided about the periphery of the captured image.

12. The method as claimed in claim 9, wherein movement detected in a third part ROI and subsequently detected in an adjacent subject's ROI, causes the motion reading from the subject's ROI to be de-weighted until the movement ceases.

13. The method as claimed in claim 1, wherein the automated monitoring apparatus includes an image detector.

14. The method as claimed in claim 11, wherein a normalized measure of motion power for both the subject's ROI regions and third party ROI regions.

15. The method as claimed in claim 4, wherein said motion determination is performed using block comparison algorithm.

16. The method as claimed in claim 15, wherein said block comparison algorithm provides a single scalar index P(t), given by:
$$P ( t ) = {\sum }_{x = 1}^{m} {\sum }_{y = 1}^{n} {{D}_{t} ( x , y )}^{2}$$
calculated from the sum power difference over successive captured image frames.

17. The method as claimed in claim 16, wherein P(t) is normalized with respect to the maximum attainable P(t) value.

18. The method as claimed in claim 4, wherein said determination of motion between captured image frames, or between ROI images is performed utilizing normalized correlation coefficients.

19. The method as claimed in claim 18, wherein said correlation coefficient r_{K} for a given region k is given by
$${r}_{k} ( t + 1 ) = \frac{{\sum }_{xy} \{ ( {f}_{t} ( x , y ) = {\overline{f}}_{t} ) ( {f}_{t} + 1 ( x , y ) - {\overline{f}}_{t} + 1 ) \}}{\sqrt{\{ {\sum }_{xy} {( {f}_{t} ( x , y ) - {\overline{f}}_{t} )}^{2} \} \times \{ {\sum }_{xy} {( {f}_{t} + 1 ( x , y ) - {\overline{f}}_{t} + 1 )}^{2} \}}} .$$

20. The method as claimed in claim 19, wherein a coefficient of determination, Rₖ=r²ₖ.

21. The method as claimed in claim 19, wherein a motion-related agitation index is defined as Aₖ(t+1)=−rₖ(t+1)²=1−Rₖ(t+1).

22. The method as claimed in claim 21, wherein a single motion-related agitation index is calculated from the captured image frame-to-frame correlation coefficients r_{K} for both subject and third-party ROI motions using fuzzy mathematics.

23. The method as claimed in claim 2, wherein for a patient subject under nursing medical supervision, a patient agitation value on said agitation index is given by at least one of the following rules, wherein;

| Rule | Patient-motion | Nurse-motion | agitation |
|---|---|---|---|
| 1. | low | low | low |
| 2. | medium | low | medium |
| 3. | high | low | high |
| 4. | low | medium | low |
| 5. | medium | medium | high |
| 6. | high | medium | high |
| 7. | low | high | medium |
| 8. | medium | high | high |
| 9. | high | high | high |

24. The method as claimed in claim 1, wherein said automated monitoring of at least one metric of a subject's autonomic nervous system (ANS) includes monitoring power spectral density (PSD) of both HRV and BPV.

25. The method as claimed in claim 1, wherein said step of quantifying agitation include;
(QRS peak detection and R-R interval calculation) and/or (systolic and diastolic blood pressure values detection)
spectral estimation and calculation of PSD in VLF, LF, and HF frequency bands and
determination of subject agitation from changes in signal dynamics.

26. The method as claimed in claim 25, wherein said ORS peak detection and R-R interval calculation detected using a Haar wavelet.

27. The method as claimed in claim 25, wherein said spectral estimation and calculation of power in VLF, LF, and HF frequency bands is performed using frequency domain analysis.

28. The method as claimed in claim 27, wherein said frequency bands are defined as high (HF) 0.15-0.4 Hz; low (LF) 0.07-0.14 Hz; very low (VLF) 0.0033-0.04 Hz.

29. The method as claimed in claim 25, wherein said spectral analysis of R-R and/or systolic blood pressure signals is performed using an adaptive autoregressive (AR) spectral estimation method.

30. The method as claimed in claim 25, wherein said PSD, P_{AR}, is given by:
$${P}_{AR} = T {σ}_{∞} {\vert \frac{1}{A ( f )} \vert}^{2} .$$

31. The method as claimed in claim 25, wherein said determination of subject agitation from changes in signal dynamics is determined using a fuzzy-logic inference system (FIS).

32. The method as claimed in claim 31, wherein inputs of said FIS include the HRV ratio VLF/MF and the BPV ratio HF/VLF.

33. The method as claimed in claim 31, wherein individual agitations levels for each input signal are recorded at a plurality of time increments T2, T3, T4, . . . Tn preceding an instantaneous level T1, wherein the individual agitation levels, obtained for HRV, systolic blood pressure and BPV, are then combined in create a single agitation value according to the rules:

| Rule | T1 | T2 | T3 | T4 | Agitation |
|---|---|---|---|---|---|
| 1 | Low | — | — | — | Low |
| 2 | Medium | High | — | — | Low |
| 3 | Medium | Medium | Low | Low | Low |
| 4 | Medium | Medium | Medium | Medium | Low |
| 5 | Low | Low | Low | Low | Low |
| 6 | High | High | High | High | High |
| 7 | High | Low | Low | Low | High |
| 8 | High | Medium | Low | Low | High |
| 9 | High | Medium | Medium | Medium | Medium |

34. A system for objective assaying of agitation in an individual or subject, said system including;
automated monitoring apparatus capable of monitoring at least one metric of
a. a patient's autonomic nervous system (ANS);
b. expert systems or rules delineating other clinical events from agitation and/or
c. physical movement of one or more defined region(s) of interest (ROI) of the patient's body,
signal processing means capable of processing physiological signals associated with the monitored metric and an subject's autonomic nervous system (ANS) and/or
physical movement of one or more defined regions of interest (ROI) of the subject's body,
signal processing means capable of processing physiological signals associated with the monitored metric and
processing means capable of calculating agitation from changes in said processing physiological signals.

35. The system as claimed in claim 35, wherein said agitation calculation provides a corresponding agitation value within a defined agitation index.

36. A method of sedation administration including the steps;
objectively quantifying agitation according to the method claimed in claim 1;
inputting said quantified agitation to an automated sedation administration system,
administering defined quantities of one or more sedatives in proportion to said quantified agitation.

37. A system for sedation administration including;
said system for objectively quantifying agitation as claimed in claim 34;
an automated sedation administration system capable of receiving said quantified agitation and administering defined quantities of one or more sedatives in proportion to said quantified agitation.

38. A fatigue and/or agitation monitoring method objectively quantifying agitation according to the method claimed in claim 1, characterised in that when a user's physical movement from one or more ROI exceeds one or more upper or lower movement threshold levels, a signal is output to one or more systems including:
an audible and/or visual alarm signal,
a graphical and/or alphanumeric information display,
one or more direction and/or velocity control means of a vehicle,
audio system,
data-logging means.

39. A method of alerting nursing/medical personnel to excessive patient agitation, including the steps;
monitoring agitation in according to the method claimed in claim 1;
outputting an alarm signal when said quantified agitation exceeds one or more predetermined threshold values.

40. A system for alerting nursing/medical personnel to excessive patient agitation, including;
said system for objectively quantifying agitation as claimed in claim 34;
an alarm capable of outputting an alarm signal when said quantified agitation exceeds one or more predetermined threshold values.

41. A means of quantifying user agitation in non-medical assessment environments including the steps;
monitoring agitation in according to the method claimed in claim 1,
wherein said quantified agitation is compared to established data recorded for non-stressed individuals to provide a relative agitation index.
